(19) United States
(12) Patent Application Publication
(10) Pub. No.: US 2011/0165097 A1
(43) Pub. Date: Jul. 7, 2011

(54) COMPOSITIONS FOR PERCUTANEOUS ADMINISTRATION

(75) Inventors: Fabienne Caillet-Bois, Massongex (CH); Isabelle Rault, Segny (FR); Michel Steiger, La Tour-de-Peilz (CH)

(73) Assignee: Novartis AG, Basel (CH)

(21) Appl. No.: 13/061,980

(22) PCT Filed: Sep. 9, 2009
(86) PCT No.: PCT/EP2009/061667

§ 371 Date: Mar. 3, 2011

(30) Foreign Application Priority Data
Sep. 10, 2008 (EP) 08164057.5

(51) Int. Cl. A61K 31/465; A61K 47/32; A61K 31/137; A61K 9/12; A61K 31/4402; A61K 31/196; A61P 31/00; A61P 29/00; A61P 17/04

(52) U.S. Cl. 424/45; 514/772.4; 514/655; 514/343; 514/357; 514/567

(57) ABSTRACT

Compositions intended for the percutaneous administration of physiologically active agents, e.g. drugs or a veterinary agents, are disclosed. Sasd compositions are characterized by having an exeefenf long term efficacy due to their ability to form a long-lasting film on the skin.

The present invention relates to compositions intended for the percutaneous administration of physiologically active agents, in particular pharmaceutically active compounds (but also e.g. agents like nicotine or veterinary drugs). Throughout this document, “percutaneous” is intended to mean any route of administering a physiologically active active agent onto, into or through the skin of a subject so as to achieve one or more of a topical, local or systemic physiological effect. Local treatment of the skin is intended to also include, for example, applying such compositions to the ear, e.g. for treating otitis with e.g. antibiotics. More specifically, the invention relates to percutaneous pharmaceutical and veterinary, especially pharmaceutical, compositions with improved long term efficacy.

With conventional percutaneous formulations like creams, solutions, gels etc., it is usually necessary to administer them repeatedly, because they tend to be rubbed off or washed away over time. Obviously, what is needed is a percutaneous formulation that exhibits excellent long term efficacy.

The present invention provides film-forming compositions that can be sprayed onto the skin or rubbed in the skin. Sprayable compositions—be it solutions, thin gels or gels—are preferred, as there is no need for the patient (or user of the composition, respectively, e.g. in veterinary applications) to come in touch with the composition anymore.

Moreover, the present invention provides such beneficial film-forming compositions, which, when applied to the skin under ambient conditions, form a true film, i.e. a thin layer. Said compositions can be e.g. substantially homogenous solutions, gels or suspensions (e.g. in case of a very poorly soluble active substance). In case of a sprayable solution or gel, said films formed on the skin are typically transparent. Due to the specific components used, said films are very robust, show good waterproofness and allow high skin permeation of the physiologically active agent(s) included over a long period of time (up to several days)—the latter in case of active substances intended for and capable of penetrating the skin.

Therefore, the invention relates to a composition intended for the percutaneous administration of a physiologically active compound, which consists essentially of

(a) 0.1-20% (w/v) of at least one physiologically active compound,

(b) 0.5-30% (w/v) of a hydrophobic polymer selected from the group consisting of acrylate polymers and copolymers, methacrylate polymers and copolymers, olefinic acid amide/acid ester/acid or alcohol polymers and copolymers, and shellac,

(c) 50-99.4% (w/v) of one or more solvents selected from the group consisting of volatile, physiologically acceptable organic solvents and water, and

(d) 0-15% (w/v) of a plasticizer.

In a preferred embodiment, a plasticizer (d) is present, too, typically in an amount of 0.1-15% (w/v), In particular 2-10% (w/v). Preferred as (d) are neutral oils.

Used as physiologically active compounds (a) can be any pharmaceutical—or veterinarily—active substance suitable for percutaneous delivery. Even physiologically active compounds that are normally delivered by the oral, parenteral or rectal route, come into consideration.

As far as the physiologically active compounds (a) are capable of forming physiologically acceptable salts, prodrugs or hydrates, the latter are included by naming (a) in free, neutral form. Examples of physiologically active compounds (a) are:

Cardioactive medications, for example, organic nitrates such as nitroglycerine, isosorbide dinitrate, and isosorbide mononitrates; quinidine sulfate; procainamide; thiazides such as bendroflumethiazide, chlorothiazide, and hydrochlorothyazide; nifedipine; nicardipine; adrenergic blocking agents, such as timolol and propranolol; verapamil; diltiazem; captopril; clonidine and prazosin.

Androgenic steroids, such as testosterone, methyltestosterone and fluoxymesterone.

Estrogens, such as conjugated estrogens, esterified estrogens, estropipate, 17beta estradiol, 17beta-estradiol valerate, equilin, mestranol, estrone, estriol, 17beta-ethinyl estradiol, and diethylstilboestrol. Progestational agents, such as progesterone, 19-norprogesterone, norethindrone, norethindrone acetate, melengestrol, chlormadinone, ethisterone, medroxyprogesterone acetate, hydroxyprogesterone caproate, ethynodiol diacetate, norethynodrel, 17alpha hydroxyprogesterone, dydrogesterone, dimethisterone, ethinylestrenol, norgestrel, demegestone, promegestone, and megestrol acetate.

Drugs having an action on the central nervous system, for example sedatives, hypnotics, antianxiety agents, analgesics and anaesthetics, such as chloral, buprenorphine, naloxone, hatoperidol, fluphenazine, pentobarbital, phenobarbital, secobarbital, codeine, fentanyl, and nicotine.

Local anesthetics, e.g. lidocaine, tetracaine, dyclonine, benzocaine, dibucaine, methocaine, procaine, mepivacaine, bupivacaine, etidocaine or prilocaine.

Nutritional agents, such as vitamins, essential amino acids, and essential fats.

Anti-inflammatory agents, such as steroids, e.g. hydrocortisone, desonide, cortisone, dexamethasone, fluocinolone, triamcinolone, medrysone, prednisolone, flurandrenolide, prednisone, halcinonide, methylprednisolone, flurandrenolide, prednisone, halcinonide, methylprednisolone, fludrocortisone, corticosterone, paramethasone, betamethasone; and non-steroidal anti-inflammatory drugs, e.g. diclofenac, ibuprofen, naproxen, fenoprofen, fenbufen, flurbiprofen, indoprofen, ketoprofen, suprofen, indomethacin, piroxicam, aspirin, salicylic acid, diflunisal, methyl salicylate, phenylbutazone, sulindac, mefenamic acid, meclofenamate sodium or tolmetin.

Anti-inflammatory agents that are often used, inter alia, in veterinary medicine, e.g. triamcinolone, betamethasone, dexamethasone, isoflupredone, hydrocortisone or prednisolone.

Antihistamines, such as dimetindene, diphenhydramine, dimenhydrinate, perphenazine, triprolidine, pyrilamine, chlorcyclizine, promethazine, carbinoxamine, tripelennamine, brompheniramine, hydroxyzine, cyclizine, meclizine, clorprenaline, terfenadine, and chlorpheniramine.

Respiratory agents, such as theophylline and beta2-adrenergic agonists such as albuterol, terbutaline, metaproterenol, ritodrine, carbuterol, fenoterol, quinterenol, rimiterol, solmefamol, soterenol, and tetroquinol.

Sympathomimetics, such as dopamine, norepinephrine, phenylpropanolamine, phenylephrine, pseudoephedrine, amphetamine, propylhexedrine, and epinephrine. Miotics, e.g. pilocarpine. Cholinergic agonists, such as choline, acetylcholine, methacholine, carbachol, bethanechol, pilocarpine, muscarine, and arecoline.

Antimuscarinic or muscarinic, cholinergic blocking agents such as atropine, scopolamine, homatropine, methscopolamine, homatropine methylbromide, methantheline, cyclopentolate, tropicamide, propantheline, anisotropine, dicyclomine, and eucatropine. Mydriatics, such as atropine, cyclopentolate, homatropine, scopolamine, tropicamide, eucatropine and hydroxyamphetamine.

Psychic energizers, e.g. 3-(2-aminopropyl)indole or 3-(2-aminobutyl) indole.

Antibiotics, e.g. clindamycin, erythromycin, tetracycline, penicillin, chloramphenicol, sulfacetamide, sulfamethazine, sulfadiazine, sulfamerazine, sulfamethizole or sulfisoxazole.

Antibiotics that are often used, inter alia, in veterinary medicine, e.g. benzylpenicillin, methycyllin, ampillicin, amoxicillin, streptomycin, neomycin, tetracyclines, chloramphenicol, erythromycin, griseofulvin, thiostrepton, florfenicol, enrofloxacin, bacitracin, gentamycin, polymyxin B, chloramphenicol, marbofloxacin or framecytin.

Antiparasitizides that are often used, especially in veterinary medicine, e.g. malachite green, methylene blue, chloramine T or B, emmamectin benzoate or alpha-cypermethrin.

Anthelmintics that are often used, inter alia in veterinary medicine, e.g. arecoline. Ivermectin, praziquantel, mebendazole or thiabendazole.

Antipsoriatic agents, e.g. calcipotriol or calcipotriol/betamethasone combinations.

Antivirals, e.g. penciclovir; acyclovir or idoxuridine.

Anti-acne agents, e.g. benzoyl peroxide.

Dermatological agents, such as vitamins A and E.

Humoral agents, such as the prostaglandins, natural and synthetic, for example PGE1, PGF2alpha, and PGF2alpha, and the PGE1 analog misoprostol.

Antispasmodics, such as atropine, methantheline, papaverine, cinnamedrine, and methscopolamine.

Antidepressant drugs, such as isocarboxazid, phenelzine, tranylcypromine, imipramine, amitriptyline, trimipramine, doxepin, desipramine, nortriptyline, protriptyline, amoxapine, maprotiline, and trazodone.

Anti-diabetics, such as insulin, and anticancer drugs such as tamoxifen and methotrexate. Anorectic drugs, such as dextroamphetamine, methamphetamine, phenylpropanolamine, fenfluramine, diethylpropion, mazindol, and phentermine.

Anti-allergenics, such as antazoline, methapyrilene, chlorpheniramine, pyrilamine and pheniramine.

Tranquilizers, such as reserpine, chlorpromazine, and antianxiety benzodiazepines such as alprazolam, chlordiazepoxide, clorazeptate, halazepam, oxazepam, prazepam, clonazepam, flurazepam, triazolam, lorazepam and diazepam.

Antipsychotics, such as thiopropazate, chlorpromazine, triflupromazine, mesoridazine, piperacetazine, thioridazine, acetophenazine, fluphenazine, perphenazine, trifluoperazine, chlorprathixene, thiothixene, haloperidol, bromperidol, loxapine, and molindone.

Decongestants, e.g. xylometazoline, oxymetazoline, phenylephrine, ephedrine or naphazoline.

Antipyretics, e.g. acetylsalicylic acid or salicylamide.

Antimigraine agents, e.g. dihydroergotamine or pizotyline.

Drugs for treating nausea and vomiting, such as chlorpromazine, perphenazine, prochlorperazine, promethazine, triethylperazine, triflupromazine, and trimeprazine.

Anti-malarials, such as the 4-aminoquinolines, alpha-aminoquinolines, chloroquine, and pyrimethamine.

Anti-ulcerative agents, such as misoprostol, omeprazole, and enprostil.

Peptides and proteins, such as drugs for Parkinson's disease, spasticity, and acute muscle spasms, such as levodopa, carbidopa, amantadine, apomorphine, bromocriptine, selegiline (deprenyl), trihexyphenidyl hydrochloride, benztropine mesylate, procyclidine hydrochloride, baclofen, diazepam, dantrolene, insulin, erythropoietin and growth hormone.

Anti-estrogen or hormone agents, such as tamoxifen or human chorionic gonadotropin.

Nucleotides and nucleic acids (e.g. DNA).

Antifungals, e.g. terbinafine, naftifine, butenafine, bifonazole, clotrimazole, econazole, isoconazole, ketoconazole, miconazole, oxiconazole, sertaconazole, sulconazole, tioconazole, tolnaftate, tereonazole, amorolfine, ciclopirox or undecylenic acid.

Antifungals that are often used, inter alia, in veterinary medicine, e.g. fluconazole, ketoconazole, isoconazole, miconazole, Amphotericin B, flucytosine, terbinafine, nystatin, thiabendazol or clotrimazol.

The physiologically active compounds (a) can be present in the composition in different forms, depending on which form yields the optimum delivery characteristics. For example, they can be. in its free base or acid form, or in the form of salts, esters, or any other pharmacologically acceptable derivatives, or e.g. as components of molecular complexes.

Preferred physiologically active compounds (a) are nicotine, lidocaine, hydrocortisone, diclofenac, ibuprofen, naproxen, indomethacin, piroxicam, methyl salicylate, phenylbutazone, mefenamic acid, betamethasone, dimetindene, scopolamine, benzoyl peroxide, calcipotriol, penciclovir, acyclovir, vitamin A, vitamin E, xylometazoline, oxymetazoline, phenylephrine, ephedrine, naphazoline, terbinafine, naftifine, butenafine, bifonazole, clotrimazole, econazole, isoconazole, ketoconazole, miconazole, oxiconazole, sertaconazole, sulconazole, tioconazole, tolnaftate, terconazole, amorolfine, ciclopirox and undecylenic acid.

Especially preferred physiologically active compounds (a) are nicotine, lidocaine, hydrocortisone, diclofenac, dimetindene, scopolamine, benzoyl peroxide, calcipotriol, penciclovir, acyclovir, xylometazoline, terbinafine, tolnaftate and clotrimazole.

In a special embodiment of the invention, the compositions of the invention are devoid of antifungal agents as physiologically active compounds (a).

The hydrophobic polymer (b) typically is an acrylate polymer or copolymer, a methacrylate polymer or copolymer, an olefinic acid amide/acid ester/acid or alcohol polymer or copolymer, or shellac. The hydrophobic polymer, more preferably, is an octylacrylamide acrylate or methacrylate, such as octylacrylamide acrylate butylaminoethyl methacrylate copolymer or octylacrylamide butylaminoethyl methacrylate copolymer; an octylpropenamide acrylate copolymer, an aminoalkyl methacrylate copolymer, an ammonio methacrylate copolymer, a PVP/VA (polyvinylpyrrolidone/vinyl acetate) copolymer, PVA (polyvinyl alcohol); an alkyl monoester of PVM/MA [poly(vinyl methyl ether-maleic anhydride] copolymer, such as the butyl monoester thereof; shellac or an alkyl acrylate/methyl methacrylate copolymer.

The hydrophobic polymer (b) typically is present in an amount of from 0.5-30% (w/v) of the composition of the invention. Preferably, the hydrophobic polymer is present in an amount of 1-20%—more preferably 1-15%, especially 2-15%, and in particular 3-12%—(w/v) of the composition.

The mono-C₁-C₇-alkyl ester of methyl vinyl ether/maleic acid copolymer is also designated as C₁-C₇-alkyl ester of PVM/MA copolymer or C₁-C₇-alkyl monoester of poly(methyl vinyl ether/maleic acid). Preferred mono-C₁-C₇-alkyl esters are the ethyl, isopropyl and n-butyl monoesters, in particular the n-butyl monoester, which is e.g. available as Gantrez® ES-43S (GAF Corporation, New York, USA).,

N—C₁-C₁₂-alkyl-C₂-C₄-alkenamide/acrylate copolymer is e.g. (tert-octylacrylamide/acrylates copolymer (Dermacryl® 78).

Typically, the one or more solvents (c) are present in a total amount of 50-99.4% —preferably 80-90% and especially 65-80%—(w/v) of the total composition.

The volatile, physiologically acceptable organic solvent in (c) is e.g. a pharmaceutically acceptable solvent or a veterinarily acceptable solvent, and preferably is selected from the group consisting of C2-C4 alkanols, C1-C4 acetate, acetone, methylethylketone, diethyl ether and tert-butyl methyl ether. Even more preferred are ethanol, propanol, isopropanol and ethyl acetate. Especially ethanol and isopropanol are preferred, and in particular 95-95% (v/v) ethanol and isopropanol.

Preferably, the total amount of the one or more solvents (c) consists of 10-99.4% (w/v) of volatile, physiologically acceptable organic solvents and of 0-90% (w/v) of wafer—and especially of 10-94.4% (w/v) of volatile, physiologically acceptable organic solvents and of 5-80% (w/v) of water, of the total composition each.

In a special embodiment of the invention, the one or more solvents (c) consist of 40-94% (w/v) of volatile, physiologically acceptable organic solvents and from 5-50% (w/v) of water, of the total composition each.

In another special embodiment of the Invention, the one or more solvents (c) consist of 10-40% (w/v) of volatile, physiologically acceptable organic solvents and from 50-80% (w/v) of water, of the total composition each.

Typically, the use of water as one of the solvents (c) is an option (but no “must”) if the physiologically active compound (a) has at least some solubility in water. In such cases, the water being present is able to increase the solubility of (a) in the composition. It was found, surprisingly, that the addition of wafer to the otherwise largely hydrophobic composition does not destroy the latter, but rather that water is fully compatible with it.

As plasticizer (d), there can be used any topically acceptable (pharmaceutically or veterinarily) plasticizer known in the art. Examples are: Acetylated hydrogenated cottonseed glyceride, acetylated hydrogenated soybeen oil glycerides, acetylated hydrogenated vegetable oil glycerides, acetyl tributyl citrate, acetyl triethyl citrate, Carnauba, castor oil, cetearyl palmitate, diacetylated monoglycerides, dibutyl sebacate, diethyl phthalate, dipropylene glycol salicylate, glycerin, neutral oils, glyceryl cocoate, glyceryl tricaprate/caprylate, glyceryl triheptanoate, hydrogenated lanolin, hydrogenated tallow glyceride lactate, mono- and di-acetylated monoglycerides, octyldodecyl myristate, PEG-8, PEG-12, PEG-20, PEG-75, PEG-150, PEG-8 dilaurate, PEG-12 dioleate, PEG-60 lanolin, PEG-8 ricinoleate, PEG-20 stearate, polybutene, polyester adipate, polyethylene glycol, polyethylene glycol monomethyl ether, polyglyceryl-10 tetraoleate, PPG-2 lanolin alcohol ether, PPG-5 lanolin alcohol ether, propylene glycol, sorbitol, triacetin, tributyl citrate and triethyl citrate (PPG=polypropylene glycol, PEG=polyethylene glycol).

In particular, there come into consideration as plasticizer (d) neutral oils; polyalcohols, e.g. glycerol, polyethylene glycol, ethylene glycol or propylene glycol; sorbitol; polysorbates [=fatty acid esters of polyoxyethylene sorbitan], such as polysorbate 80 [=polyoxyethylene (20) sorbitan monooleate]; C1-C6 alkyl esters of citric acid, e.g. acetyl tributyl citrate; or dialkyl phthalates, e.g. diethyl phthalate. Preferably, (d) is a neutral oil.

A neutral oil typically is a glyceride, which means fatty acid esters of glycerine. The fatty acid components may be saturated, e.g. caprylic acid or capric acid, or unsaturated, e.g. oleic acid. Glycerides may be of natural origin, e.g. castor oil, semi-synthetic, e.g. hydrogenated castor oil, or, preferably, completely synthetic. Preferred are triglycerides, in particular those with C₆-C₁₄ saturated fatty acids, but e.g. also glycerol monoesters with C6-C18 fatty acids, e.g. n-octanoic acid or oleic acid; come into consideration.

The plasticizer (d) is an optional component but, preferably, is present in an amount of 0.1-15%—more preferably 2-10%, especially 3-8% and in particular 4-6%—(w/v) of the total composition.

Typically, the percutaneous compositions of the invention are either liquids or viscous liquids, in some instances they may also be In gel form. Preferably, they are in sprayable form, and can be applied e.g. as a pump spray or as an aerosol spray, the latter typically being sealed and further including a propellant. Especially, they are in sprayable form as such, i.e. sprayable without use of e.g. a propellent. In other words, they are applied in the form of a spray (without use of e.g. a propellant), e.g. as pump sprays.

In another embodiment of the invention, the percutaneous compositions are suitable to be rubbed on the skin, especially in the form of a gel or viscous liquid.

Moreover, the percutaneous compositions of the invention may optionally contain usual percutaneously acceptable non-essential excipients known in the art.

Permeation enhancers, e.g. oleyl alcohol or cineol, may optionally be added to ensure effective permeation of the active substance to the desired target location, in a manner known per se.

pH regulators may optionally be added to adjust the pH of the composition to a desired value. Examples for pH regulators are triethanolamine, ethanolamine, triethylamine, diethylamine or specific buffer mixtures, e.g. NaH₂PO₄×2H₂O/anhydrous Na₂HPO₄.

Other optional non-essential excipients known in the art include e.g. chelating agents and isotonicity regulators, surfactants, antioxidants and UV absorbers.

Another embodiment of the invention relates to compositions intended for the percutaneous administration of a physiologically active agent, which composition comprises

(a) 0.1-20% (w/v) of at least one physiologically active compound,

(b) a hydrophobic polymer being either 1-30% (w/v) of a mono-C₁-C₇-alkyl ester of methyl vinyl ether/maleic acid copolymer or 0.5-25% of N—C₁-C₁₂-alkyl-C₂-C₄-alkenamide/acrylates copolymer, and

(c) 10-98% (w/v) of at least one volatile, physiologically acceptable organic solvent, and

(d) 0-80% (w/v) of water;

with the proviso that it is devoid of any hydrophilic polymer and thickening agent, and with the further proviso that it is devoid of any antifungal agent.

As outlined above, the percutaneous compositions of the invention show inter alia excellent long term efficacy, mechanical robustness and waterproofness as well as a high skin permeation of the drug, as far as desired, over a long period of time. Said beneficial properties can be demonstrated e.g. by the following tests:

(1) The mechanical properties of the films are tested by, in particular, measuring the tensile strength, the Young's modulus and the elongation of the film. Moreover, the films are tested e.g. in a shear test, a stress relaxation or a elastic deformation test.

(2) Film robustness is determined e.g. by oscillating a piece of gauze over glass slides on which 100 mg of a test composition have been evenly spread and allowed to dry at 50° C. for 10 min.

(3) Specific properties related to the application of the compositions of the invention that are tested are their spreadability, their resistance to water and their skin adhesion.

(4) Waterproofness is determined e.g. by evenly spreading a test composition on glass slides, allowing to dry It and weighing the glass slide with the dried film. The glass slides are immersed in a beaker of deionized water at 20° C. for 20 min. Then they are removed, dried in an oven at 50° C. and weighed again. Waterproofness is calculated from the weights of the glass slides before and after water treatment.

(5) In vitro skin retention of drug component: The skin levels of the drug are determined after application of the test composition on the skin surface after 24 h and within the epidermis after 24 h. in vitro diffusion cells using excised human epidermis are used, The test composition is applied to epidermal membrane and the amount of drug penetrating subsequently measured (HPLC and UV detection).

The compositions of the invention can be manufactured in a manner known per se, for example by conventional mixing and homogenization methods.

The following examples illustrate the invention.

### EXAMPLES

Manufacturing method of Example 1 (for a batch of 1 liter), exemplary for all other examples: Introduce 0.4 kg of ethanol (aqueous, 96%) into a dissolutor, add 50 g of octylacrylamide/acrylates copolymer under stirring and continue to stir until dissolution will be complete. Add neutral oil, oleyl alcohol and stir until homogeneity. Add diclofenac diethylammonium salt and stir until dissolution will be complete. Put the solution in a 1 l volumetric flask (glass) and adjust until the gauge with ethanol (aqueous, 98%). Stir for 15 minutes.

if a hydrophobic polymer (b) other than octylacrylamide/acrylates copolymer is used, e.g. n-butyl monoester of PVM/MA copolymer, the process is analogous to the one described above.

If e.g. nicotine bitartrate is used as physiologically active substance (a), it is first solubilized in e.g. ethanol, then are added, one after the other, (b), (d) and the buffer solution, and finally the volume is adjusted.

### Example 1

### Sprayable Film-Forming Solution Comprising 4.65% (w/v) of Diclofenac Diethylammonium Salt

| | |
|---|---|
| Diclofenac diethylammonium salt | 4.65% |
| (corresponding to 4% of Diclofenac Na) | |
| Octylacrylamide/acrylates copolymer (Dermacryl ® 79) | 5% |
| Neutral oil (medium chain triglycerides, mainly caprylic/ | 5% |
| capric acid triglyceride, Miglyol ® 812) | |
| Oleyl alcohol | 2% |
| Ethanol (aqueous, 96%) | 68.4% |

### Example 1a

### Sprayable Film-Forming Solution Comprising 1% (w/v) of Diclofenac Na

| | |
|---|---|
| Diclofenac Na | 1% |
| Octylacrylamide/acrylates copolymer (Dermacryl ® 79) | 5% |
| Neutral oil (medium chain triglycerides, mainly caprylic/ | 5% |
| capric acid triglyceride, Miglyol ® 812) | |
| Ethanol (aqueous, 96%) | 72.3% |

### Example 1b

### Sprayable Film-Forming Solution Comprising 1% (w/v) Diclofenac Na

| | |
|---|---|
| Diclofenac Na | 1% |
| Octylacrylamide/acrylates copolymer (Dermacryl ® 79) | 5% |
| Neutral oil (medium chain triglycerides, mainly caprylic/ | 5% |
| capric acid triglyceride, Miglyol ® 812) | |
| Isopropanol | 69.93% |

### Example 1c

### Sprayable Film-Forming Solution Comprising 4% (w/v) Of Diclofenac Na

| | |
|---|---|
| Diclofenac Na | 4% |
| Octylacrylamide/acrylates copolymer (Dermacryl ® 79) | 5% |
| Neutral oil (medium chain triglycerides, mainly caprylic/ | 5% |
| capric acid triglyceride, Miglyol ® 812) | |
| Ethanol (aqueous, 96%) | 70.8% |

### Example 2

Sprayable film-forming solution comprising 4% (w/v) of Diclofenac Na: Same composition as in Example 1, but with 4% Diclofenac Ha and 68.8% of Ethanol instead of 4.65% Diclofenac diethylammonium salt and 68.4% Ethanol.

| | |
|---|---|
| Diclofenac Na | 4% |
| Octylacrylamide/acrylates copolymer (Dermacryl ® 79) | 5% |
| Neutral oil (medium chain triglycerides, mainly caprylic/ | 5% |
| capric acid triglyceride, Miglyol ® 812) | |
| Oleyl alcohol | 2% |
| Ethanol (aqueous, 96%) | 68.8% |

### Example 2a

Sprayable film-forming solution comprising 4% (w/v) of Diclofenac Na: Same composition as in Example 2, but with 2% Cineol and 69% of Ethanol instead of 2% Oleyl alcohol and 68.8% of Ethanol.

| | |
|---|---|
| Diclofenac Na | 4% |
| Octylacrylamide/acrylates copolymer (Dermacryl ® 79) | 5% |
| Neutral oil (medium chain triglycerides, mainly caprylic/ | 5% |
| capric acid triglyceride, Miglyol ® 812) | |
| Cineol | 2% |
| Ethanol (aqueous, 96%) | 69% |

### Example 3

### Sprayable Film-Forming Solution Comprising 4.65% (w/v) of Diclofenac Diethylammonium Salt

| | |
|---|---|
| Diclofenac diethylammonium salt | 4.65% |
| (corresponding to 4% of Diclofenac Na) | |
| Octylacrylamide/acrylates copolymer (Dermacryl ® 79) | 5% |
| Triethanolamine | 1.61% |
| Oleyl alcohol | 2% |
| Ethanol (aqueous, 96%) | 20% |
| Water | 65.1% |

### Example 4

Sprayable film-forming solution comprising 4% (w/v) of Diclofenac Na: Same composition as in Example 3, but with 4% Diclofenac Na and 65.1% of water instead of 4.65% Diclofenac diethylammonium salt and 65.1% water.

| | |
|---|---|
| Diclofenac Na | 4% |
| Octylacrylamide/acrylates copolymer (Dermacryl ® 79) | 5% |
| Triethanolamine | 1.61% |
| Oleyl alcohol | 2% |
| Ethanol (aqueous, 96%) | 20% |
| Water | 65.1% |

### Example 5

### Sprayable Film-Forming Solution Comprising 0.5% (w/v) of Dimetindene Maleate

| | |
|---|---|
| Dimetindene maleate | 0.5% |
| Gantrez ® ES-435 | 10% |
| Neutral oil (medium chain triglycerides, mainly caprylic/ | 5% |
| capric acid triglyceride, Miglyol ® 812) | |
| Triethanolamine | 2.5% |
| Ethanol (aqueous, 96%) | 66.3% |

### Example 8

Sprayable film-forming solution comprising 0.5% (w/v) of Dimetindene maleate: Same composition as In Example 5, but with 1% (w/v) of Ethanolamine and 67% of Ethanol instead of 2.5% (w/v) Triethanolamine and 66.3% Ethanol,

| | |
|---|---|
| Dimetindene maleate | 0.5% |
| Gantrez ® ES-435 | 10% |
| Neutral oil (medium chain triglycerides, mainly caprylic/ | 5% |
| capric acid triglyceride, Miglyol ® 812) | |
| Ethanolamine | 1% |
| Ethanol (aqueous, 96%) | 67% |

### Example 7

Sprayable film-forming solution comprising 0.1% (w/v) of Dimetindene maleate: Same composition as in Example 5, but with 0.1% (w/v) of Dimetindene maleate, 1.1% (w/v) of Ethanolamine and 66.8% of Ethanol instead of 0.5% (w/v) of Dimetindene maleate, 2.5% (w/v) Triethanolamine and 66.3% Ethanol.

| | |
|---|---|
| Dimetindene maleate | 0.1% |
| Gantrez ® ES-435 | 10% |
| Neutral oil (medium chain triglycerides, mainly caprylic/ | 5% |
| capric acid triglyceride, Miglyol ® 812) | |
| Ethanolamine | 1.1% |
| Ethanol (aqueous, 96%) | 66.8% |

### Example 8

Sprayable film-forming solution comprising 0.5% (w/v) of Dimetindene maleate: Same composition as in Example 5, but with 1.7% (w/v) of Triethylamine and 66.1% of Ethanol instead of 2.5% (w/v) Triethanolamine and 66.3% Ethanol.

| | |
|---|---|
| Dimetindene maleate | 0.5% |
| Gantrez ® ES-435 | 10% |
| Neutral oil (medium chain triglycerides, mainly caprylic/ | 5% |
| capric acid triglyceride, Miglyol ® 812) | |
| Triethylamine | 1.7% |
| Ethanol (aqueous, 96%) | 66.1% |

### Example 9

Sprayable film-forming solution comprising 0.1% (w/v) of Dimetindene maleate: Same composition as in Example 5, but with 0.1% (w/v) of Dimetindene maleate, 1.5% (w/v) of Triethylamine and 66.5% of Ethanol instead of 0.5% (w/v) of Dimetindene maleate, 2.5% (w/v) Triethanolamine and 66.3% Ethanol.

| | |
|---|---|
| Dimetindene maleate | 0.1% |
| Gantrez ® ES-435 | 10% |
| Neutral oil (medium chain triglycerides, mainly caprylic/ | 5% |
| capric acid triglyceride, Miglyol ® 812) | |
| Triethylamine | 1.5% |
| Ethanol (aqueous, 96%) | 66.5% |

### Example 10

Sprayable film-forming solution comprising 0.5% (w/v) of Dimetindene maleate: Same composition as in Example 5, but with 1.1% (w/v) of Diethylamine and 66.7% of Ethanol instead of 2.5% (w/v) Triethanolamine and 66.3% Ethanol.

| | |
|---|---|
| Dimetindene maleate | 0.5% |
| Gantrez ® ES-435 | 10% |
| Neutral oil (medium chain triglycerides, mainly caprylic/ | 5% |
| capric acid triglyceride, Miglyol ® 812) | |
| Diethylamine | 1.1% |
| Ethanol (aqueous, 96%) | 66.7% |

### Example 11

Sprayable film-forming solution comprising 0.1% (w/v) of Dimetindene maleate: Same composition as in Example 5, but with 0.1% (w/v) of Dimetindene maleate, 1% (w/v) of Diethylamine and 67.7% of Ethanol instead of 0.5% (w/v) of Dimetindene maleate, 2.5% (w/v) Triethanolamine and 66.3% Ethanol.

| | |
|---|---|
| Dimetindene maleate | 0.1% |
| Gantrez ® ES-435 | 10% |
| Neutral oil (medium chain triglycerides, mainly caprylic/ | 5% |
| capric acid triglyceride, Miglyol ® 812) | |
| Diethylamine | 1% |
| Ethanol (aqueous, 96%) | 67.7% |

### Example 12

### Sprayable Film-Forming Solution Comprising 0.5% (w/v) of Dimetindene Maleate

| | |
|---|---|
| Dimetindene maleate | 0.5% |
| Octylacrylamide/acrylates copolymer (Dermacryl ® 79) | 5% |
| Neutral oil (medium chain triglycerides, mainly caprylic/ | 5% |
| capric acid triglyceride, Miglyol ® 812) | |
| Ethanol (aqueous, 96%) | 72.6% |

### Example 13

Sprayable film-forming solution comprising 0.1 % (w/v) of Dimetindene maleate: Same composition as in Example 12. but with 0.1% Dimetindene maleate and 72.8% of Ethanol instead of 0.5% Dimetindene maleate and 72.6% Ethanol.

| | |
|---|---|
| Dimetindene maleate | 0.1% |
| Octylacrylamide/acrylates copolymer (Dermacryl ® 79) | 5% |
| Neutral oil (medium chain triglycerides, mainly caprylic/ | 5% |
| capric acid triglyceride, Miglyol ® 812) | |
| Ethanol (aqueous, 96%) | 72.8% |

### Example 14

### Sprayable Film-Forming Solution Comprising 0.45% (w/v) of Nicotine Bitartrate

| | |
|---|---|
| Nicotine bitartrate | 0.45% |
| Octylacrylamide/acrylates copolymer (Dermacryl ® 79) | 5% |
| Neutral oil (medium chain triglycerides, mainly caprylic/ | 5% |
| capric acid triglyceride, Miglyol ® 812) | |
| Buffer solution (to reach pH 8.2) | 2.7% |
| [prepared from 0.6 g NaH₂PO₄ × 2H₂O | |
| and 13.64 g anhydrous Na₂HPO₄ in 1 liter of water] | |
| Ethanol (aqueous, 96%) | 71.2% |

### Example 15

### Sprayable Film-Forming Solution Comprising 0.15% (w/v) of Nicotine Free Base

| | |
|---|---|
| Nicotine free base | 0.15% |
| Octylacrylamide/acrylates copolymer | 5% |
| (Dermacryl ® 79) | |
| Miglyol ® 812 | 5% |
| Ethanol (aqueous, 96%) | 69% |

### Example 16

### Sprayable Film-Forming Solution Comprising 0.5% (w/v) of Nicotine Free Base

| | |
|---|---|
| Nicotine free base | 0.5% |
| Octylacrylamide/acrylates copolymer (Dermacryl ® 79) | 5% |
| Neutral oil (medium chain triglycerides, mainly caprylic/ | 5% |
| capric acid triglyceride, Miglyol ® 812) | |
| Ethanol (aqueous, 96%) | 62.7% |

### Example 17

### Sprayable Film-Forming Solution Comprising 0.5% (w/v) of Nicotine Free Base

| | |
|---|---|
| Nicotine free base | 0.5% |
| Gantrez ® ES-435 | 10% |
| Neutral oil (medium chain triglycerides, mainly caprylic/ | 5% |
| capric acid triglyceride, Miglyol ® 812) | |
| Ethanol (aqueous, 96%) | 67.8% |

### Example 18

Sprayable film-forming solution comprising 0.45% (w/v) of Nicotine bitartrate; Same composition as in Example 17, but with 0.45% of nicotine bitartrate and 72.6% of Ethanol instead of 0.15% of nicotine free base arid 69% Ethanol.

| | |
|---|---|
| Nicotine bitartrate | 0.45% |
| Octylacrylamide/acrylates copolymer (Dermacryl ® 79) | 5% |
| Neutral oil (medium chain triglycerides, mainly caprylic/ | 5% |
| capric acid triglyceride, Miglyol ® 812) | |
| Ethanol (aqueous, 96%) | 72.6% |

### Example 19

### Sprayable film-forming solution comprising 1.125% (m/v) of Terbinafine HCl

| | |
|---|---|
| Terbinafine HCl | 1.125% |
| Gantrez ® ES-435 | 10% |
| Neutral oil (medium chain triglycerides, mainly caprylic/ | 5% |
| capric acid triglyceride, Miglyol ® 812) | |
| Ethanol (aqueous, 96%) | 61.2% |

### Example 20

### Sprayable film-forming solution comprising 1.125% (w/v) of Terbinafine HCl

| | |
|---|---|
| Terbinafine HCl | 1.125% |
| Octylacrylamide/acrylates copolymer (Dermacryl ® 79) | 5% |
| Neutral oil (medium chain triglycerides, mainly caprylic/ | 5% |
| capric acid triglyceride, Miglyol ® 812) | |
| Ethanol (aqueous, 96%) | 72% |

1. A composition intended for percutaneous administration of a physiologically active compound, which consists essentially of
(a) 0.1-20% (w/v) of at least one physiologically active compound,
(b) 0.5-30% (w/v) of a hydrophobic polymer selected from the group consisting of acrylate polymers and copolymers, methacrylate polymers and copolymers, olefinic acid amide/acid ester/acid or alcohol polymers and copolymers, and shellac,
(c) 50-99.4% (w/v) of one or more solvents selected from the group consisting of volatile, physiologically acceptable organic solvents and water, and
(d) 0-15% (w/v) of a plasticizer.

2. A composition according to claim 1, wherein the amount of plasticizer (d) is 0.1-15% (w/v).

3. A composition according to claim 1, wherein the hydrophobic polymeric (b) is selected from the group consisting of 1-30% (w/v) of a mono-C₁-C₇-alkyl ester of methyl vinyl ether/maleic acid copolymer or 0.5-25% of N—C₁-C₁₂-alkyl-C₂-C₄-alkenamide/acrylates copolymer.

4. A composition according to claim 3, wherein the hydrophobic polymeric (b) is a n-butyl monoester of methyl vinyl ether/maleic acid copolymer or an octylacrylamide/acrylates copolymer.

5. A composition according to claim 3, wherein the hydrophobic polymeric (b) is an octylacrylamide/acrylates copolymer.

6. A composition according to claim 1, wherein the hydrophobic polymer (b) is present in an amount of 2-15% (w/v) of the total composition.

7. A composition according to claim 1, wherein the one or more solvents (c) consist of 10-94% (w/v) of volatile, physiologically acceptable organic solvents and from 5-90% (w/v) of water, of the total composition each.

8. A composition according to claim 7, wherein the one or more solvents (c) consist of 40-94% (w/v) of volatile, physiologically acceptable organic solvents and from 5-50% (w/v) of water, of the total composition each.

9. A composition according to claim 7, wherein the one or more solvents (c) consist of 10-40% (w/v) of volatile, physiologically acceptable organic solvents and from 50-80% (w/v) of water, of the total composition each.

10. A composition according to claim 1, wherein a volatile, physiologically acceptable organic solvent (c) is selected from the group consisting of C2-C4 alkanols, C1-C4 acetate, acetone, methylethylketone, diethyl ether and tert-butylmethyl ether.

11. A composition according to claim 1, wherein the amount of water in (c) is less than 5% (w/v).

12. A composition according to claim 1, wherein the one or more solvents (c) are selected from the group consisting of 95-97% (v/v) ethanol and isopropanol.

13. A composition according to claim 1, wherein the at least one physiologically active compound (a) is selected from the group consisting of nicotine, lidocaine, hydrocortisone, diclofenac, ibuprofen, naproxen, indomethacin, piroxicam, methyl salicylate, phenylbutazone, mefenamic acid, betamethasone, dimetindene, scopolamine, benzoyl peroxide, calcipotriol, penciclovir, acyclovir, vitamin A, vitamin E, xylometazoline, oxymetazoline, phenylephrine, ephedrine, naphazoline, terbinafine, naftifine, butenafine, bifonazole, clotrimazole, econazole, isoconazole, ketoconazole, miconazole, oxiconazole, sertaconazole, sulconazole, tioconazole, tolnaftate, terconazole, amorolfine, ciclopirox and undecylenic acid.

14. A composition according to claim 1, which is in sprayable form as such, i.e. without use of e.g. a propellant.

15. A composition according to claim 2, wherein the hydrophobic polymeric (b) is selected from the group consisting of 1-30% (w/v) of a mono-C₁-C₇-alkyl ester of methyl vinyl ether/maleic acid copolymer or 0.5-25% of N—C₁-C₁₂-alkyl-C₂-C₄-alkenamide/acrylates copolymer.

16. A composition according to claim 15, wherein the hydrophobic polymeric (b) is a n-butyl monoester of methyl vinyl ether/maleic acid copolymer or an octylacrylamide/acrylates copolymer.

17. A composition according to claim 16, wherein the hydrophobic polymeric (b) is an octylacrylamide/acrylates copolymer.

18. A composition according to claim 17 wherein the plasticizer comprises a neutral oil.

19. A composition according to claim 17 which additionally comprises a permeation enhancer.

20. A composition according to claim 16 wherein a volatile, physiologically acceptable organic solvent (c) is selected from the group consisting of C2-C4 alkanols, C1-C4 acetate, acetone, methylethylketone, diethyl ether and tert-butylmethyl ether.

21. A composition according to claim 17, wherein the one or more solvents (c) are selected from the group consisting of 95-97% (v/v) ethanol and isopropanol.

22. A composition according to claim 1 wherein which is in sprayable form and applied as an aerosol spray, the latter being sealed and further including a propellant.

23. A composition according to claim 2 wherein which is in sprayable form and applied as an aerosol spray, the latter being sealed and further including a propellant.
